(19) United States
(12) Patent Application Publication
(10) Pub. No.: US 2009/0131802 A1
(43) Pub. Date: May 21, 2009

(54) Contact sensor for fiberoptic raman probes

(75) Inventors: Stephen Fulghum, Marblehead, MA (US); Charles Von Rosenberg, Belmont, MA (US); Jonathan Feld, Somerville, MA (US); Sudha Thimaraju, Andover, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: Newton Laboratories Incoporated, Belmont, MA (US)

(21) Appl. No.: 12/291,545

(22) Filed: Nov. 10, 2008

Related U.S. Application Data
(60) Provisional application No. 61/002,723, filed on Nov. 9, 2007.

(51) Int. Cl. A61B 6/00

(52) U.S. Cl. 600/478

(57) ABSTRACT

The present invention relates to an optical contact sensor for a spectroscopic probe. The sensor detects contact of the distal end of a fiber optic probe to a surface being measured. The system can be used to correct Raman spectral measurements of tissue.

### CROSS REFERENCE TO RELATED APPLICATIONS

The present application claims priority to U.S. Provisional Patent Application No. 61/002,723 filed Nov. 9, 2007. The entire contents of the above application is incorporated herein by reference.

### BACKGROUND OF THE INVENTION

Fiberoptic Raman spectroscopy probes used in conjunction with autofluorescence endoscopes can improve the specificity for cancer detection compared to using autofluorescence alone. Autofluorescence in human tissue is generally blue to blue-green when excited with UV to violet wavelengths. Precancerous and cancerous tissue does not fluoresce as strongly as normal tissue and thus shows up in the visual field of the endoscope as a relatively dark patch in a brighter field. The method is very sensitive but not always specific for cancer. Fiberoptic Raman probes can be passed through the biopsy channel of the endoscope and pressed against these darker areas of tissue for an independent spectral diagnosis. The combination of the two diagnoses is more reliable than either one alone.

Raman scattering is sensitive to the concentrations of specific chemicals in the tissue which change when the tissue becomes cancerous. In the Raman process some energy is absorbed from an incident photon and converted into vibrational motion of the molecules so that the scattered photon is red-shifted in wavelength. With narrowband incident excitation, such as from a laser, the result is a “fingerprint” of narrow lines representing different molecules in the tissue with different vibrational energies. The scattering process is quite weak, however, so the amount of the Raman scattered light is low. The Raman peaks are typically superimposed on a larger background of broadband, relatively featureless tissue fluorescence. Raman scattering at near-infrared (NIR) wavelengths is particularly useful since the background noise due to tissue fluorescence is lower at NIR wavelengths than at visible wavelengths.

Most optical tissue diagnostic algorithms require comparisons of an acquired spectrum with a similar spectrum from a different tissue site, perhaps taken earlier in the same patient from a site known to be normal. Spectra are often compared to a large set of spectra which have been correlated with pathology results during the development of those algorithms. Sometimes the absolute amplitude of the overall spectrum does not matter But that is not generally the casein the lung, for instance, the broad, featureless tissue fluorescence that complicates Raman spectra is typically stronger in normal lung tissue than it is in dysplastic lung tissue. Diagnostics which depend on relative signal comparisons like this require a stable measurement system and repeatable probe placement techniques.

The design of practical Raman fiberoptic probes makes repeatable probe placement onto the tissue particularly important. Particular lens can result in a rapid falloff in collection efficiency with increasing distance from the tissue. Fractions of a millimeter in probe to tissue spacing can significantly change the level of the acquired signal. This can make diagnostics which depend on relative measurements less reliable if contact is uncertain.

The low intensity of the Raman scattered light means that the white light illumination of the endoscope's visual field must be reduced or turned off during Raman data acquisition. Longer wavelength light leaking into the probe can add noise to the Raman spectra. The low signal levels also result in typical data acquisition times of 1 second or longer. Low illumination and long acquisitions add to the clinician's difficulty in properly placing the probe, maintaining probe position and maintaining probe contact. Involuntary tissue motion and tissue folds which may hide the tip of the Raman probe further complicate the positioning of these probes.

### SUMMARY OF THE INVENTION

The present invention describes a method and apparatus by which effective contact between a fiberoptic, spectroscopic probe and tissue can be verified and monitored both before and during the acquisition of spectroscopic data from a tissue area being probed. Effective contact is preferred for the relative comparison of measurements between different tissue sites and for comparisons with the previously-acquired data sets used in diagnostic programs. A monitoring system for probe contact can thus be used to indicate the likely reliability of a given diagnostic result or its information may be used to normalize integrated spectral signatures back to standard levels when probe contact is occasionally lost during a long integration. These normalized signatures can be used for a more reliable diagnosis rather than being discarded.

In a preferred embodiment the monitor system couples light into the excitation fiber at the proximal end of a fiberoptic probe along with any illumination or excitation light required by the probe for spectroscopic purposes. The fiberoptic probe delivers the combined monitor and excitation light to the tissue at the distal end of the probe. A fraction of the monitor light scattered from the tissue is collected by the probe and returned to its proximal end by means of a number of collection fibers along with the desired spectroscopic signature. This returned monitor light is separated from the light required for spectroscopic purposes and passed to a photodetector to be quantified. Since the monitor light is relatively strong relative to the Raman signal a single collection fiber can also be dedicated to the contact monitor and coupled directly to a photodetector.

The monitor light returning from the distal tip of a probe consists of two components. The first component is due to Fresnel reflections from the glass/air interface at the distal tip of the probe itself. This Fresnel component will decrease in contact with a water or a wet absorbing surface due to the reduced reflectivity of a glass/water interface. The second component is due to diffuse reflection from tissue near the distal tip and drops off rapidly with increasing distance of the probe tip from the tissue. The relative changes in the size of these two components depend upon the specific design of the optics at the distal tip of the probe, the wavelength of the light used for the contact monitor and the nature of the tissue surface. Either or both signals may be used to detect probe contact.

For a standard, forward-looking Raman probe with a ball lens at the distal tip the tissue reflectivity signal dominates the Fresnel reflection signal upon tissue contact. This is particularly true if the monitor light source is chosen with a wavelength which is not absorbed significantly by hemoglobin in the tissue (>630 nm) and if the photodetector optics are designed to reject the higher angle light reflected from the concave surface at the distal end of the probe. As a probe is slowly lowered onto tissue with a thick water layer the signal will have an initial value followed by a somewhat lower value as the tip touches the water surface followed by a significantly higher value when the tip finally touches tissue.

For forward-looking Raman probes designed with flat optical surfaces at the distal tip the falloff of the diffuse reflection signal with tissue distance is less rapid. For side-looking Raman probes designed to be inserted through biopsy needles the tissue may remain very close to the probe window with a narrow air gap that should be avoided. In these cases it may be advantageous to maximize the Fresnel reflection relative to the diffuse tissue reflection and detect a reduction in the signal upon contact with a water film on the tissue surface. The monitor wavelength chosen for this case may be one where hemoglobin absorption is stronger, such as 532 nm or 405 nm, to further minimize the tissue reflectivity.

In either case, a good estimate for a signal reference level to determine contact or no contact can be obtained by prior testing of a particular type of probe on accessible mucosal tissue such as the hand or lip. A histogram of signal levels can also be readily maintained during a particular procedure by continuously updating how often a particular monitor signal level is obtained. Initially this level represents the background signal from the distal tip of the probe in air. The first contact with tissue increases the histogram count at higher signal levels. The transition between contact and no contact is fast for forward-looking probes so that relatively few histogram counts are obtained in the transition zone. An adaptive algorithm can determine the optimal reference signal level by determining a value which is roughly equidistant between the first peak on the low side of the histogram and the first peak on the high side of the histogram, regardless of whether the algorithm is looking for a signal increase or decrease.

For a standard ball lens probe, the background signal due to the Fresnel reflection component can either be measured and subsequently subtracted during signal processing or it can be reduced by proper spatial filtering of the light exiting the collection fibers at the proximal end of the fiberoptic probe. In the ball lens Raman probe, for example, the Fresnel reflection comes from the concave final surface of the lens which strongly focuses the reflected light so that it enters the collection fibers at a very steep angle. This steep angle is essentially maintained through many internal reflections within the collection fibers so that this component exits the collection fibers at a similar steep angle. When the light exiting the collection fibers is collimated by the first lens in the spectrometer this first component of monitor light shows up as a bright ring at the outer edge of the collimated beam (the Fourier transform of high angle light). A spatial filter in this nominally collimated beam that passes low angle light nearer the center of the collimated beam can reject most of this first component. This is desirable since it reduces the sensitivity of the later monitor signal processing software processing to variations in the intensity of the monitor light source and to variations in the collection efficiency between different probes of the same configuration.

Besides the monitor light returning to the proximal photodetector there may also be additional light due to endoscope illumination and/or tissue fluorescence which happens to be at the wavelength of the monitor light source. Two methods are used to reduce this external background signal. When the monitor light source is a narrowband laser a narrow bandpass optical filter can be placed in front of the monitor signal photodetector to reject out-of-band light. Generally this first method eliminates most, but not all, of the background light. The second method is to pulse the monitor light source and record the photodetector signal both with the monitor source on and with the monitor source off. If these two measurements are taken close together in time compared to typical intensity fluctuations in the background light (say at measurement rates of 10 Hz or above) the difference between these two measurements is the desired monitor light signal due to diffuse tissue reflection (or Fresnel reflection).

Pulsing the monitor light signal has an additional advantage when the contact monitor system is used with video endoscopes. Some of the monitor light will generally be visible in the video image of the endoscope system either because the probe is not in contact or because the monitor light is transmitted through the tissue when the probe is in contact. The integrated intensity of the monitor light source during a single video frame must not be too great or the camera pixels will be saturated. By pulsing the light for a brief period of time the monitor source can be used at its maximum value for the optimum measurement signal to noise ratio while avoiding camera saturation. The differential contact measurement can be made within a few milliseconds compared to the 33 millisecond video frame period so significant attenuation of the monitor light in the video image is possible.

The video image of the monitor light is also useful for correct positioning of the fiberoptic probe during spectroscopic measurement. Given that Raman signals are very weak the endoscope lighting may be significantly attenuated or even absent during Raman measurements. In the case of the fiberoptic Raman probe the monitor light exits the distal tip of the probe in a relatively narrow beam. The spot of monitor light projected onto the tissue surface can be used to guide the probe to the proper spot on the tissue surface. Since the monitor light is preferentially from a narrowband source such as a laser it is easily filtered out of the light collected for spectroscopic purposes. Typical endoscope illumination, on the other hand, is derived from an arc lamp source with a large amount of NIR power relative to the Raman signals even after extensive optical filtering.

The choice of wavelength for the monitor light must take into consideration the detailed design of the probe it is used with. In the case of the fiberoptic Raman probe the monitor light passes through two sets of filters located at the distal tip of the Raman probe. The first filter is located at the distal end of the Raman excitation delivery fiber. This filter blocks the (longer wavelength) Raman-scattered light generated within the excitation fiber but passes the power from the Raman excitation laser source. The second filter is placed before the collection fibers to block Raman excitation light reflected from the tissue and probe tip while passing the longer wavelength Raman-scattered light from the tissue being probed. This Raman excitation light can create an additional background signal in the long glass path leading back to the spectrometer. The specific wavelength that works for a particular Raman filter set may change from one type of probe to another but long-wavelength monitor laser sources are inexpensive and several different monitor lasers can be coupled separately into the delivery fiber and switched in appropriately. Diode lasers can also be temperature tuned over several nm by varying the power to the thermoelectric coolers typically used to stabilize them.

### BRIEF DESCRIPTION OF THE DRAWINGS

Preferred embodiments of the present invention are described with reference to the following drawings:

FIG. 1 is a schematic diagram of the probe contact monitor system with a fiberoptic Raman probe showing the optical components used to separate the monitor light reflected from the tissue from the Raman scattering and the means for quantifying this monitor light.

FIG. 2 is a schematic diagram of optical rays traced through a model of the distal tip of a fiberoptic Raman probe showing the nominally collimated beam exiting the probe tip as it illuminates the tissue and the detector surface which quantifies the light collected by the probe.

FIG. 3 is a graphical illustration of the light collected by the probe from diffusely scattering tissue as a function of the probe distance from the tissue. The fixed background collected from specular reflections off of the ball lens tip is also shown.

FIG. 4 is a graphical illustration of an actual signal from a probe contact monitor of the above embodiment as it is placed into contact with human mucosal tissue three times over the course of a few seconds.

FIG. 5 is a histogram of the signal shown in FIG. 4 showing that an adaptive decision algorithm can choose an optimal threshold value for contact determination by analyzing a signal histogram which is continuously updated during the course of a clinical procedure.

FIG. 6 is a diagram of optical rays traced through a model of the distal tip of a fiberoptic Raman probe showing that specular reflections from the ball lens at the tip of the probe enter the probe collection fibers at steep angles.

FIG. 7 shows the far-field distribution on a detector due to specular reflect of light from the ball lens exiting the collection fibers at the proximal end of the probe and the spatial filter aperture which can prevent most of this light from reaching a detector.

FIG. 8 shows the equivalent far-field distribution on a detector due to diffusely reflected light from tissue exiting the collection fibers indicating that much of it can pass through the same aperture shown in FIG. 7.

FIGS. 9A-9D shows the wavelength transmission characteristics of the optical filters at the tip of a typical Raman probe and the range of monitor light wavelengths which pass through both filters effectively.

FIG. 10 shows a flow chart of a preferred method for acquiring data for normalizing measured spectra using the contact monitor system.

FIG. 11 is a graphical illustration of hemoglobin absorption versus wavelength which indicates preferred wavelength range for laser sources for emphasizing tissue reflectance (>630 nm) or minimizing tissue reflectance (<450 nm).

FIG. 12 shows the contact monitor signal versus tissue distance function for a forward-looking ball lens probe and a forward-looking half ball lens probe.

FIG. 13 shows a typical path of Fresnel reflected light through a forward-looking probe with a flat exit face perpendicular to the probe axis for contact monitor light introduced through a collection fiber rather than through the central excitation fiber.

FIG. 14 shows a typical path for Fresnel reflected light from a tilted, flat exit face for contact monitor light introduced through the central excitation fiber.

FIG. 15 shows a typical path for Fresnel reflected light from the cylindrical exit surface of a side-looking Raman probe that can be used, for example, for insertion through a biopsy needle.

FIG. 16 shows the Raman fiberoptic probe cable utilizing the central delivery fiber to carry both the Raman light source and contact monitor light source and one collection fiber bundle returning both the Raman scattering signal and the contact monitor signal as shown in more detail in FIG. 1.

FIG. 17 shows an embodiment of the fiber cable system using, for example, the probe shown in FIG. 13, in which a collection fiber is used to collect in the contact monitor light and the received contact monitor light is split off from the received Raman scattered light inside the spectrometer.

FIG. 18 shows an embodiment of the fiber cable system using, for example, the probe design shown in FIG. 13, in which a collection fiber is used to collect the contact monitor light and a separate nominal collection fiber is used to return the collected contact monitor light directly to a light sensor, such as, a photodetector.

FIG. 19 shows an embodiment of a fiber cable system using, for example, the probe design in FIG. 14 or FIG. 15, in which the contact monitor light is multiplexed with the Raman excitation light for delivery through the central delivery fiber and a single collection fiber is used to return the contact monitor signal directly to a photodetector.

### DETAILED DESCRIPTION OF THE INVENTION

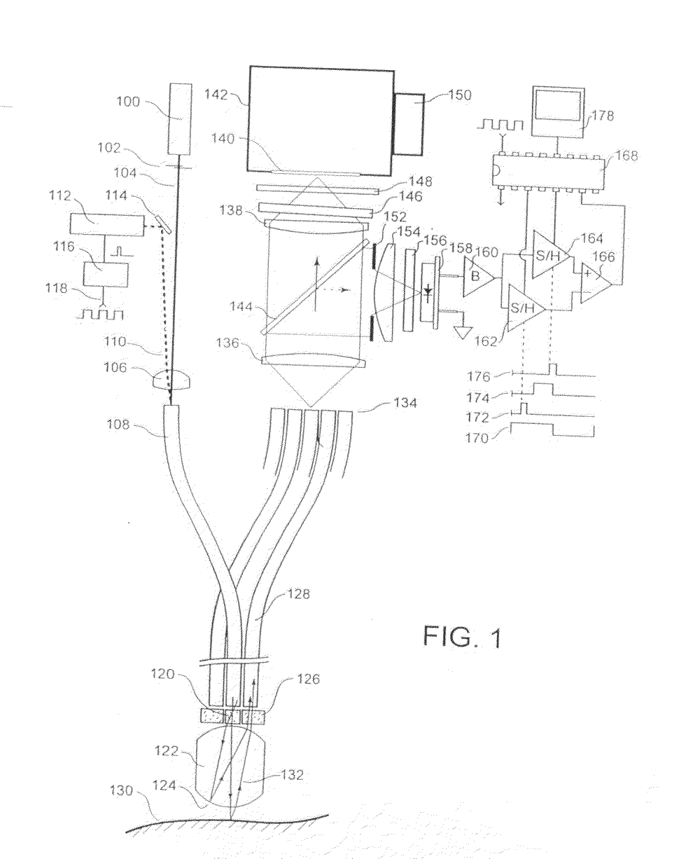

A preferred embodiment of the invention is illustrated in FIG. 1 which shows a schematic diagram of the contact monitor system as implemented with a fiberoptic Raman probe. The Raman excitation laser 100 can be run continuously but its beam 102 is pulsed on during measurements by shutter 104. The excitation beam is then coupled into the Raman probe delivery fiber 106 by a lens 108.

The relatively weak probe contact monitor system beam 110 from its laser source 112 is angularly multiplexed into the delivery fiber 106 by directing it into the coupling lens 108 at a shallow angle with scraper mirror 114. The monitor system laser is pulsed electronically with circuit 116 at the appropriate time as determined from a video synchronization pulse 118 which can be derived from the endoscope video monitor signal.

Both the Raman excitation light 102 and the probe contact monitor light 110 are carried to the distal tip of the Raman probe through the delivery fiber 106. They both pass through the Raman rod filter 120 which rejects long-wavelength Raman shifted light generated in the delivery fiber 106. Both beams then enter the ball lens (or drum lens) 122. A small quantity of each beam is reflected where they intersect the ball lens exit surface 124 and a small portion of this reflected light passes through the Raman filter 126 before entering the probe collection fibers 128. This filter 126 can be a ring filter that has characteristics to block the Raman excitation source wavelength to prevent background Raman signals from being generated in the long collection fibers 128. The contact monitor system wavelength, however, is preferably chosen so that much of it passes through this second Raman filter. Further details regarding a Raman probe system can be found in U.S. application Ser. No. 10/407,923, filed on Apr. 4, 2003, the entire contents of which is incorporated herein by reference.

The ball lens 122 focuses most of the Raman excitation light and contact monitor light onto the tissue surface 130. Some of the resulting Raman-scattered light from the tissue and some of the diffusely scattered contact monitor light 132 is refocused by the ball lens 122 and coupled back into the collection fibers 128 after passing through the Raman ring filter 126. Most of the Raman excitation light is only diffusely scattered by the tissue (and thus not wavelength-shifted) and is blocked by the Raman ring filter 126.

The Raman scattering process immediately randomizes the direction of the Raman-scattered photons with the unscattered excitation photons generally continuing deeper into the tissue. The monitor light photons, however, are redirected by diffuse scattering to exit the tissue and be collected by a light collection system. The monitor light photons are typically at shorter wavelengths and will thus scatter faster, essentially simulating the Raman-scattered photons in terms of their collection versus probe-to-tissue distance. Most of the use of the contact monitor probe is in terms of on/off collection during the data acquisition period since the transition is very fast. The intermediate stage can be measured on representative mucosal tissue for a more precise correlation of their relative signals as a function of probe-to-tissue distance.

The collection fibers at the proximal end of the fiberoptic Raman probe 134 are aligned, bonded and polished. The polished ends are imaged with lenses 136 and 138 onto the entrance slit 140 of the Raman spectrometer 142. The first lens 136 collimates the beams exiting the collection fibers and a dichroic beamsplitter 144 is used as an optical separator which reflects the visible portion of the collected light and passes the NIR portion to the spectrometer to separate the monitor and diagnostic signals. Before entering the spectrometer a high quality, narrowband rejection filter 146 reduces the intensity of the remaining Raman excitation light by five to six orders of magnitude. A red glass absorbing filter 148 rejects the remaining broadband visible light and passes the red-shifted Raman scattered light and tissue fluorescence to the spectrometer. A CCD camera 150 records the spectra of this light for later analysis and tissue diagnosis.

The monitor light reflected off of dichroic filter 144 is passed through an aperture 152 which rejects most of the angle light reflected from the ball lens at the distal tip of the Raman probe. A laser line filter 156 passes the monitor light but blocks most of the broadband light from the endoscope white light illumination or tissue fluorescence induced by the autofluorescence endoscope. The remaining monitor light and background light at the same wavelength is passed on to photodiode 158 to be measured.

The monitor light signals are pulsed but do not need to be measured at very high frequencies so the photodiode 158 can be used in the photovoltaic or zero-biased mode for the lowest noise. A buffer circuit 160 utilizes a very large feedback resistor and a low bias current operational amplifier to convert the photodiode current to a voltage followed by low-pass filtering stages before the signal is finally measured. The signal is measured before the monitor laser source 112 is turned on by a sample-and-hold circuit and analog-to-digital converter 162 and after the monitor light source has stabilized by an equivalent circuit 164. The difference between these two measurements is taken by differencing circuit 166 to eliminate the effect of more slowly-varying background light. These measurement and timing circuits may be analog and discrete or their functions may be conveniently performed within a single programmable microcontroller 168. This microcontroller can also provide the discrimination of the resulting monitor signal with the reference threshold to determine a binary contact/no contact signal or as well as implement the adaptive histogram method of determining the optimal reference threshold for a given patient.

The microcontroller can also provide the timing pulses required by the contact monitor system which are all referenced to the video synchronization square wave 170 determined externally from the video signal of the autofluorescence endoscope. This synchronization is identical to the sync pulse 118 called out elsewhere in the figure. The monitor laser pulse can be triggered in either the odd or even video field for a 29.97 Hz update rate. The trigger to perform the background measurement 172 is followed by the signal to turn on the monitor light source 174 and the signal to perform the monitor+background measurement 176.

The final result of the contact monitor is presented to the clinician with visual display 178 which may be either a visible light or a visible mark on the autofluorescence video monitor. The result is also recorded so that it can be included in the processing of the measured Raman/fluorescence signal. A Raman signal in which the probe maintained contact for 90% of the integration time can be successfully renormalized by processing with the monitor signal to what it would have been with 100% contact during the integration time.

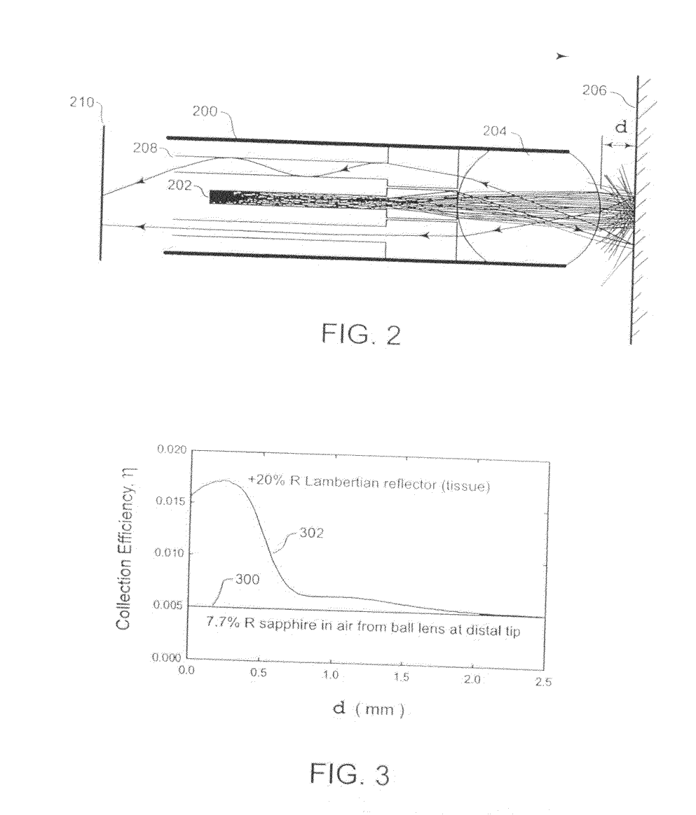

FIG. 2 is a diagram of light rays traced through a optical model of the distal tip of a fiberoptic Raman probe 200, which can be a tube having a diameter of less than 3 mm, and preferably 2 mm or less. When the probe is in air, the light rays 202 entering the central delivery fiber exit the ball lens 204 in a nominally collimated beam before reaching the tissue surface 206. The resulting spot of light is observed by the clinician in the visual field of the autofluorescence endoscope and indicates the point on the tissue that the probe is approaching. In a low-light illumination situation suitable for Raman data acquisition this beam can be the only illumination available for positioning the probe. Since the monitor light is typically visible and narrowband, however, it is easily rejected by the filters in the Raman spectrometer.

The detailed optical model of FIG. 2 was used to generate the graph of the collection efficiency for monitor light as a function of probe distance from the tissue, d. This graph is shown in FIG. 3. The unvarying value 300 represents the result of a 7.7% specular reflection from the sapphire/air interface of the ball lens when the probe is not in tissue contact. The curve which varies with distance 300 is the collection efficiency of the probe at different distances from the tissue which has been assumed to be a 20% Lambertian reflector. The important point to note in this graph is that the efficiency of collection drops rapidly beyond a separation of about 0.5 mm from the tissue.

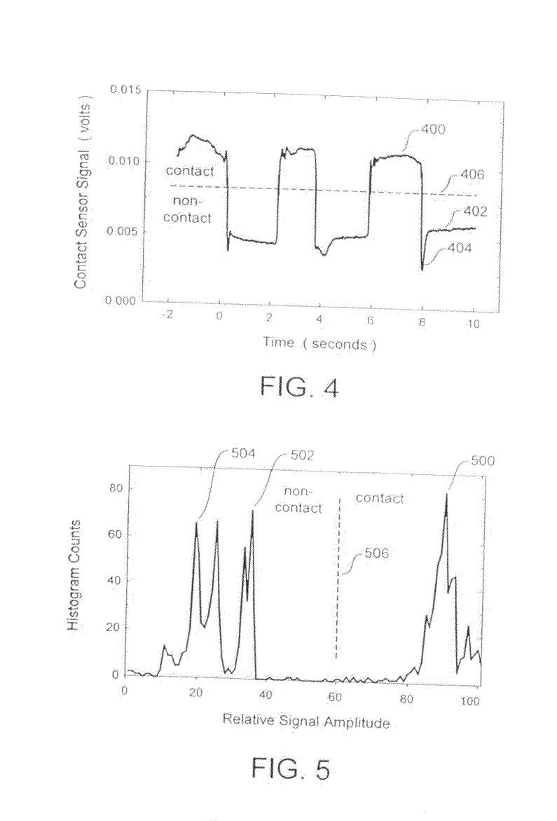

FIG. 4 is a graphical illustration of a signal from a probe contact monitor of the above as it is placed into contact with human mucosal tissue three times over the course of about 12 seconds. The peak of the signal at 400 represents good tissue contact. The lower value 402 represents the signal from a probe in air. The lowest signal level represents to probe in the thin fluid interface on the mucosal surface. The dotted signal level 406 represents a reasonable threshold value for considering the probe to be in or out of tissue contact.

FIG. 5 is a histogram of the signal levels shown in FIG. 4. for the entire 12 second period. The difference between the minimum signal and the maximum signal has been divided into 100 intervals and the number of discrete measurements falling into those intervals has been calculated. The peak 500 at about 90% represents the most common signal in tissue contact. The peak 502 at about 35% represents a high value of the signal with the probe in air and the multiple peaks 504 represent the signal when the probe just touches the fluid interface over the mucosal tissue. The dotted line 506 at 60% represents a median distance between the two largest peaks and is equivalent to the threshold 406 in FIG. 4. All of these peaks will move with variations in the monitor light source power which should thus be kept constant. The higher peak 500 representing tissue contact will change with tissue reflectivity.

Hemoglobin is the primary absorbing species in tissue so the preferred choice for a monitor light source is a diode laser with a wavelength greater than 600 nm and preferably greater than 630 nm where hemoglobin absorption is low. Since tissue reflectivity will vary with the patient, with the type of tissue being probed and with the presence or absence of blood on the tissue surface an adaptive algorithm is desirable. Minimizing the background signal of the probe in air will also increase the reliability of the contact measurement. Histogram analysis can be performed on a long rolling list of the most recent contact measurements to adapt to changing tissue types or tissue states during the procedure.

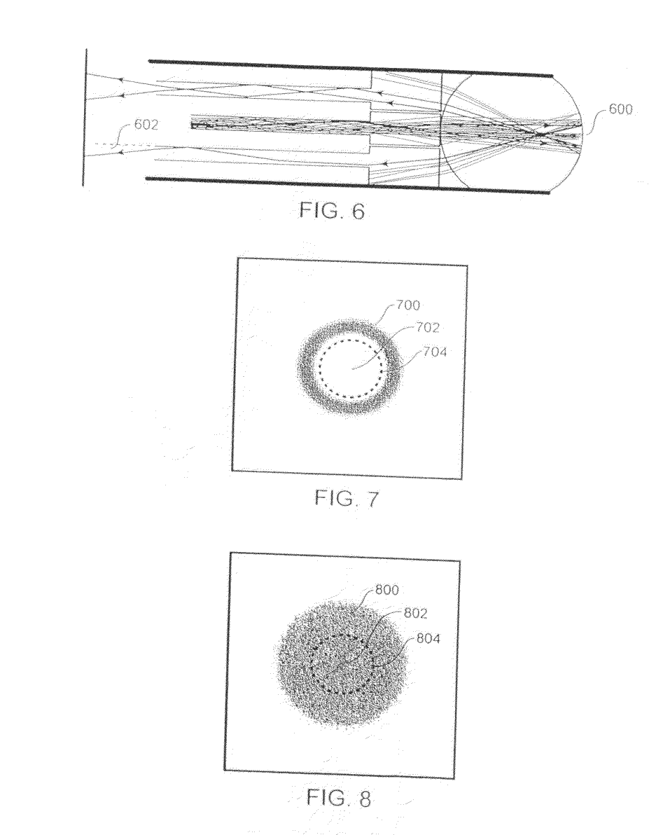

FIG. 6 is a diagram of light rays traced through the detailed model the Raman probe showing how specular reflections from the exit surface of the ball lens at the tip of the probe 600 come to a focus inside of the ball lens 602 which is not at the optimum point for the most efficient collection. The rays which are collected 604 enter the collection fibers at a steep angle which is maintained as these rays propagate back to the proximal end of the collection fibers.

FIG. 7 shows the far-field distribution on a detector of the specular reflected light from the ball lens which exits the collection fibers at the proximal end of the probe. The far field distribution is also the distribution in the finite diameter collimated beam produced by lens 136 in FIG. 1. The high angle rays from the specular reflection at the distal tip of the probe form a bright ring 700 with relatively little power on the collimated beam axis 702. An aperture with diameter 704 blocks most of this background light from reaching the contact monitor detector.

FIG. 8 shows the equivalent far-field distribution on a detector for the diffusely reflected light from the tissue which exits the collection fibers at the proximal end of the probe. The intensity distribution 800 in the collimated beam is essentially circular and uniform with significant intensity 802 on the collimated beam axis. Much of the total diffuse reflection power is transmitted through the aperture 804 while most of the specular reflection power is blocked.

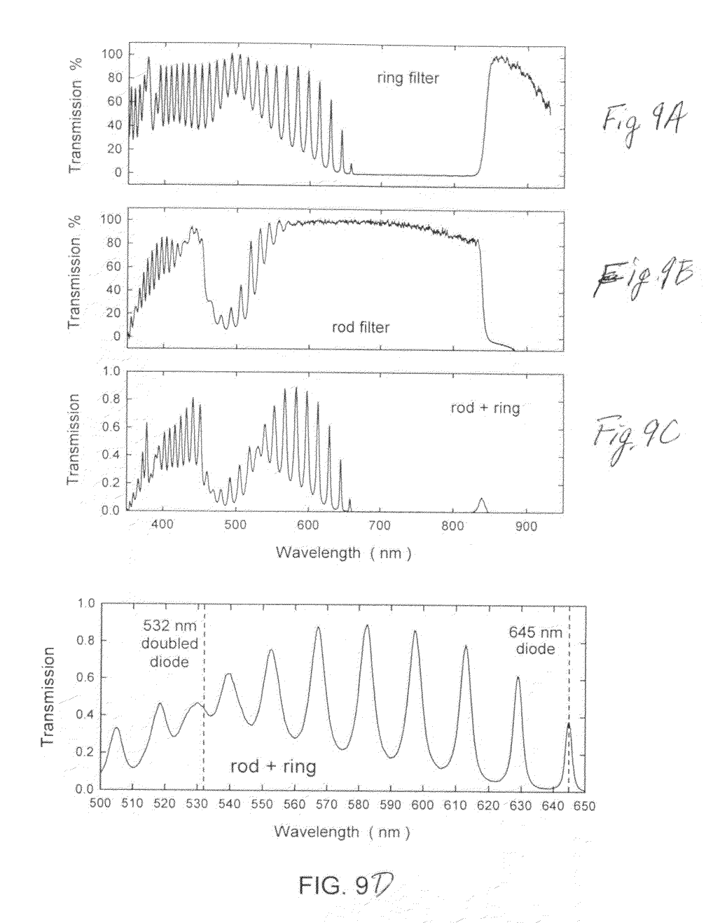

FIGS. 9A-9D show the wavelength transmission characteristics of the optical filters at the tip of a typical Raman probe. The product of the two transmission curves in FIG. 9A (ring filter) and 9B (rod filter) is the combined transmission through both filters (FIG. 9C). These particular filters were not controlled for out-of-band transmission but can be controlled for this purpose. For this filter set a (FIG. 9D) standard diode laser at 645 nm is passed efficiently and is not strongly absorbed by blood in the tissue or on the tissue surface. A doubled-diode laser at 532 nm is also transmitted very well, is relatively inexpensive and can be used when more tissue absorption is desired. Violet diode lasers at 405 nm and shorter wavelengths can be used when a high tissue absorption is required for emphasizing Fresnel reflection. Different wavelengths, can be combined off-axis in the central delivery fiber connector to accommodate different Raman probe designs.

Even though the contact monitor is particularly useful for Raman spectroscopic probe the system can be used for visible fluorescence probes and visible diffuse reflectance probes as well. In this case the monitor laser can be chosen from diode lasers with wavelengths between 670 and 780 nm which can be seen visually or by video endoscopes but still be outside the range of most fluorescence and diffuse fluorescence diagnostics.

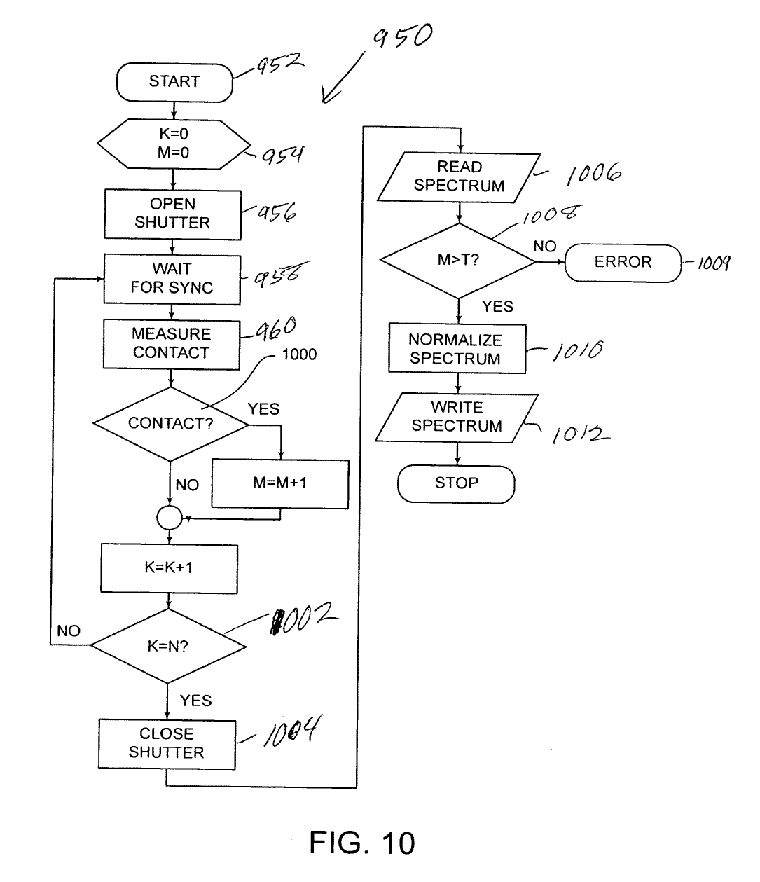

FIG. 10 shows a process sequence 950 for the acquisition of a Raman spectrum for a total period of N video frames along with N measurements using the contact monitor system. After the user initiates a measurement 952, the frame counter 954 is K and M is the counter for those measurements determined to be in contact. T is a threshold value less than or equal to N which will be equal to N for full contact during the total acquisition period. The shutter 956 is opened, and after synchronization 958, a measurement 960 is performed. The counter for M or K is adjusted depending upon the measured outcome. If the sequence of measurements is completed 1002, the shutter is closed 1004. The spectrum is read 1006 and if M is less than the threshold T previously determined to be the minimum number of frames necessary to be acquired for a reliable measurement, then a measurement error 1009 is announced to the user or clinician. If M is greater than the threshold T then the acquired spectrum can be reliably normalized 1010 and the spectrum is written 1012. A simple normalization factor is N/M which means that if all measurements were in contact, no normalization is required. More precise normalizations are possible using a transfer function determined from measurements of the relative efficiency of a contact monitor measurement and a Raman scattering spectrum measurement for varying tissue to probe distances.

The contact decision algorithm 1000 embedded in the flow chart determines whether or not the good contact counter M is incremented following any single measurement. This algorithm may look for either increased or decreased contact monitor signal depending on the design of the Raman probe in use and can use adaptive modifications to the contact/no contact threshold for the contact signal determined by the most recent histogram of contact measurements. The contact decision algorithm may also consider the stability of the contact signal over a number of recent contact measurements <=N by weighting past measurements before changing the state of the contact/no contact decision. This is effectively equivalent to limiting the frequency bandwidth of the contact measurement.

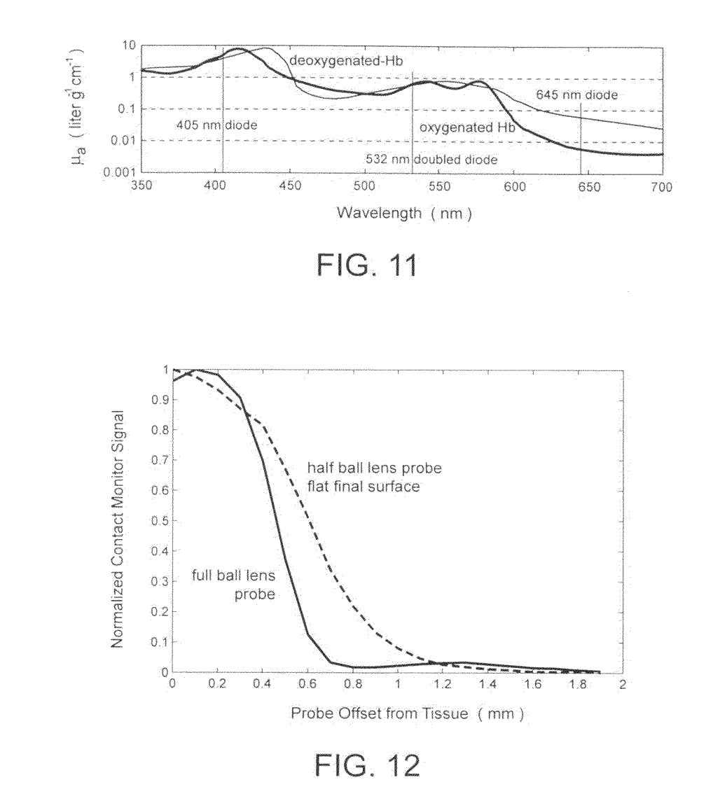

FIG. 11 shows a graph of the absorption coefficient of hemoglobin as a function of wavelength. Both the oxygenated and de-oxygenated states of hemoglobin are shown. The actual absorption can depend on both the oxygenation state of the tissue and its hemoglobin concentration as well as the thickness of any blood layer on the tissue surface. Generally blue wavelengths are absorbed strongly and red wavelengths are easily transmitted. For a forward-looking, ball lens probe a relativly long wavelength in a range above 600 nm, such as 645 nm or 658 nm, is preferable for the contact monitor light source so that tissue variation has less effect on the signal than tissue distance.

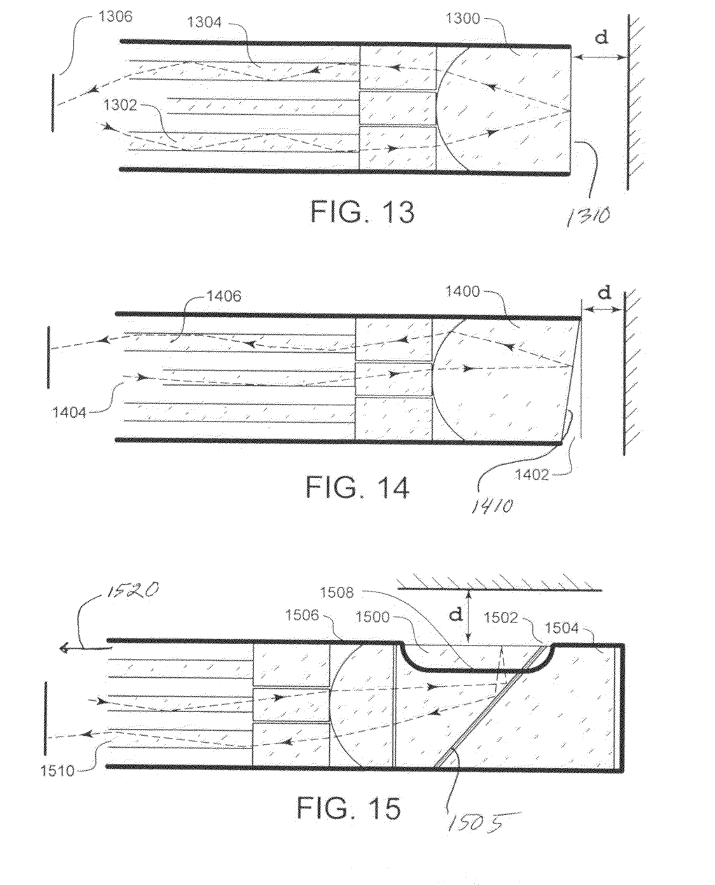

FIG. 12 shows a representation of contact monitor diffuse reflection signal levels as a function of tissue distance for both a ball lens probe and a half ball lens probe with a flat exit surface. Generally the flat exit surface reduces the steepness of the diffuse reflection signal transition and thus reduces the sensitivity of the contact sensor. Flat probe tips, however, generally increase the depth sensitivity of the Raman probe which may be important in some applications. Raman probes with flat (or cylindrical) exit surfaces can be optimized to be very sensitive to Fresnel reflections which can recover or increase the effectiveness of the contact monitor system. FIGS. 13 through 15 show preferred embodiments of the probe tip surface.

FIG. 13 shows a Raman probe built with a half ball lens 1300 with a flat exit surface perpendicular to the axis 1308 of the probe. A typical path for a Fresnel reflection is traced for light introduced through a collection fiber 1302 on the periphery of the probe rather than through the central delivery fiber. When the exit surface 1310 of the lens 1300 is placed at the nominal focus of the lens the light exiting the input collection fiber is imaged efficiently into the collection fiber 1304 on the opposite side of the probe. This means that a very strong Fresnel reflection signal can be recorded at the proximal end of the probe with a photodetector 1306 coupled directly to the collection fiber 1304. The drawback of this design is that a least one and perhaps two (out of typically 10 to 12) collection fibers are dedicated to the contact monitor rather than to Raman signal collection.

FIG. 14 shows another embodiment on the partial ball lens which uses a lens 1400 with a exit surface 1410 tilted at a small angle 1402 which is preferably in a range of 2-12 degrees, typically about 8 degrees. This design focuses the contact monitor light exiting the central fiber 1404 onto a single collection fiber 1406. This embodiment allows angular multiplexing to be used to combine the Raman excitation light and the contact monitor light, saving a collection fiber for the Raman signal. The imaging in this embodiment is as good as FIG. 13 so that the collection of the Fresnel reflected light is very efficient. Separating the collection fiber carrying the contact monitor light from the Raman scattering collection bundle increases the complexity of the probe bundle and adds another probe connector, but also simplifies the optical design of the spectrometer optics which can reduce the overall cost of the system.

FIG. 15 shows a side-looking Raman probe optimized for insertion through a hollow biopsy needle. In this application, the tissue is always close to the side window of the probe but may not be in intimate contact. Typically a vacuum 1520 can be pulled on such a probe assembly, which can be done using a pump or wall suction connected to a channel extending from the proximal to the distal end of the probe to bring the tissue into contact with the probe. A contact monitor, for this case, looks for the existence of a high Fresnel reflection indicating an unwanted air gap between the probe and the tissue. In this embodiment, the optical element 1500 which directs the view sideways is a cyindrical optical glass rod with polished edges whose back face is cut and polished at a nominal angle between 30 and 50 degrees, preferably about 40 to 45 degrees. This back face 1505 is coated with a metallic film 1502 to ensure reflectivity at all incidence angles. A second, identical element 1504 is epoxied to the first element 1500 to form a solid cylinder for insertion into a cylindrical carrying tube 1506. A portion 1508 of the carrying tube is cut away to allow the light to pass in and out of the probe. Since the filters, lenses and beam directors are all cylindrical, the space between the carrying tube 1506 and the components can be filled with epoxy to form the hermetic seal necessary for a probe to be used in surgical procedures. By design, the angle of the back face 1505 can be but slightly less than 45 degrees, typically 40 degrees, to focus the Fresnel reflected light predominately onto the lower collection fibers. A selected collection fiber 1510 is used to carry the contact monitor signal back directly to a photodetector separate from the Raman scattering collection bundle.

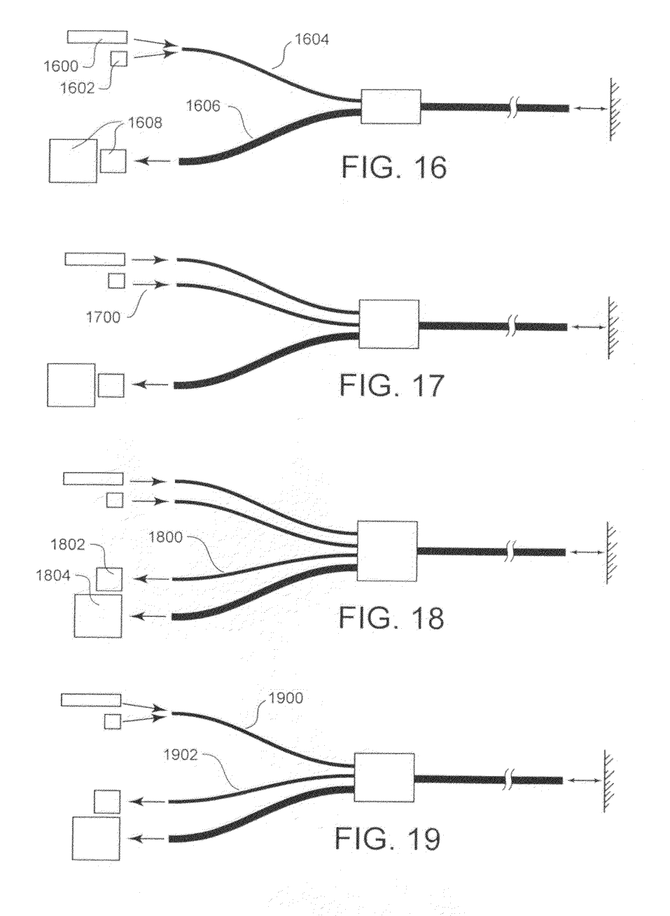

FIGS. 16 through 19 are diagrams of how the optical fibers can be bundled and epoxied into connectors for implementing embodiments of the Raman probe.

FIG. 16 is the bundling system for a standard Raman probe system shown in FIG. 1. This embodiment couples the Raman excitation light source 1600 and the contact monitor light source 1602 into the central delivery fiber 1604, returns all of the signals through a single collection fiber bundle 1606 and separates the contact monitor light from the Raman scattered light inside of a combined photodetector/spectrometer 1608 as shown in FIG. 1.

FIG. 17 shows how the optical fibers can be bundled for the probe shown in FIG. 13 in which the contact monitor light source is delivered through a peripheral collection fiber separate from the central delivery fiber. This method requires the addition of an additional single fiber 1700 and connector.

FIG. 18 shows how the optical fibers can be bundled for the probe shown in FIG. 13 when a collection fiber 1800 is separated from the Raman collection bundle and dedicated to the delivery of the collected contact monitor light to a separate photodiode 1802 which is separate from the spectrometer 1804.

FIG. 19 is a preferred embodiment which shows how the optical fibers are bundled to effect the probe shown in FIG. 14 and FIG. 15. Both the Raman excitation light and the contact monitor light are multiplexed into the central delivery fiber 1900. This embodiment utilizes one additional output fiber bundle 1902 and connector than the basic design of FIG. 16, but simplifies the optical system in the spectrometer by eliminating the need for (and the losses from) the dichroic beamsplitter in the collimated filter path as shown in FIG. 1. This embodiment can be used for both a Raman probe, which emphasizes Fresnel reflection for the contact monitor, and a probe which emphasizes tissue reflection for the contact monitor.

While the present invention has been described herein in conjunction with a preferred embodiment, a person with ordinary skill in the art, after reading the foregoing specification, can effect changes, substitutions of equivalents and other types of alterations to the system or method as set forth herein. Each embodiment described above can also have included or incorporated therewith such variations as disclosed in regard to any or all of the other embodiments. Thus, it is intended that protection granted by Letters Patent hereon be limited in breadth and scope only by definitions contained in the appended claims and any equivalents thereof.

What is claimed is:

1. A system for monitoring a fiberoptic probe in the collection of spectroscopic data from a surface comprising:
a light source at the proximal end of the probe and a coupler that couples light from the light source into a probe delivery fiber for delivering to the surface;
a light collection system that collects monitor light returning from the surface;
a detector that detects the collected monitor light; and
a processing system that determines probe contact to the surface from the detected monitor light.

2. The system of claim 1 wherein the processing system includes a computer program that normalizes integrated spectroscopic information acquired by the probe over a period of time to correct for intermittent probe contact using a value determined from one or more measurements of the monitoring light during an integration period.

3. The system of claim 1 wherein the processing system adaptively determines an optimal reference quantity based on one or more current measurements.

4. The system of claim 1 further comprising a spatial filter that reduces the quantity of returned monitor light measured in the absence of probe contact.

5. The system of claim 1 wherein the monitor light source is pulsed to allow differential measurements of returned light at the monitor wavelength with the monitor source on and off to provide background subtraction.

6. The system of claim 1 wherein the monitor light source is visible to an operator or a video camera to provide illumination for the placement of the spectroscopic probe.

7. The system of claim 1 wherein the processing system compares a quantity of collected monitoring light and a reference quantity.

8. The system of claim 1 wherein the monitor source is a laser.

9. The system of claim 1 wherein at least one of a plurality of monitor laser sources is selectively used to work with a spectroscopic probe.

10. The system of claim 1 wherein the light collection system comprises a plurality of collection optical fibers that collects reflected monitor light and Raman light from the tissue and an optical separator coupled to a proximal end of the optical fibers that separates the collected reflected light to the detector and the collected Raman light to a second detector.

11. The system of claim 1 wherein the system further comprising a light source emitting light having a wavelength greater than 600 nm for obtaining Raman spectroscopic data.

12. A method of determining probe contact at a tissue surface comprising:
illuminating a tissue surface with light from a distal end of a probe;
collecting light returning from the tissue surface with the probe; and
determining whether the distal end of the probe is in contact with the tissue surface.

13. The method of claim 12 further comprising using a probe for insertion within an animal body.

14. The method of claim 12 further comprising detecting Raman spectroscopic data from the tissue surface.

15. The method of claim 14 further comprising at least periodically performing a contact measurement during a data acquisition period.

16. The method of claim 12 further comprising using a probe having a tubular distal body with a diameter of 3 mm or less.

17. The method of claim 12 further comprising normalizing a spectrum using a contact measurement.

18. The method of claim 12 further comprising measuring Fresnel reflections to determine contact.

19. The method of claim 12 further comprising measuring diffusely reflected light from the tissue surface to determine contact.

20. The method of claim 12 further comprising using a first light source for a contact measurement and a second light source for a second measurement.
